(19) United States
(12) Patent Application Publication
(10) Pub. No.: US 2021/0317077 A1
(43) Pub. Date: Oct. 14, 2021

(54) Crystalline Forms of N1-(1-Cyanocycloproply)-N2-((1S)-1-{4'-[(1R-2,2-Difluoro-1-Hydroxyethyl]Biphenyl-4-YL}-2,2,2-Trifluoroethyl)-4-Fluoro-L-Leucinamide

(71) Applicant: Intervet Inc., Madison, NJ (US)

(72) Inventors: Xiaoling Jin, Green Brook, NJ (US); Dirk Stueber, Union City, NJ (US); Cynthia Bazin, Quebec (CA); Rositza Iordanova Petrova, Metuchen, NJ (US); Christophe Pierre Alain Chassaing, Ingelheim am Rhein (DE); Hans Peter Niedermann, Bubenheim (DE); Stephan Veit, Budenheim (DE); Claudia Scheipers, Ingelheim (DE); Jochen Schoell, Luzern (CH)

(73) Assignee: Intervet Inc., Madison, NJ (US)

(21) Appl. No.: 17/264,417

(22) PCT Filed: Aug. 1, 2019
(86) PCT No.: PCT/EP2019/070769

§ 371 Date: Jan. 29, 2021

(30) Foreign Application Priority Data
Aug. 2, 2018 (EP) 18187052.8

(51) Int. Cl. C07C 255/46

(52) U.S. Cl. CPC C07C 255/46; C07C 2601/02; C07B 2200/13

(57) ABSTRACT

The present disclosure encompasses crystalline forms of N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide and processes for the preparation thereof.

### BACKGROUND

U.S. Pat. No. 7,407,959 B2 discloses the compound N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide and process to produce the same. The IUPAC name of this compound is (2S)-N-(1-cyanocyclopropyl)-2-[[(1S)-1-[4-[4-[(1R)-2,2-difluoro-1-hydroxy-ethyl]phenyl]phenyl]-2,2,2-trifluoro-ethyl]amino]-4-fluoro-4-methyl-pentanamide.

### BRIEF DESCRIPTION OF THE DRAWINGS

FIG. 1 is a is a characteristic X-ray diffraction pattern of the crystalline Form A.

FIG. 2 is a carbon-13 cross-polarization magic-angle spinning (CPMAS) nuclear magnetic resonance (NMR) spectrum of the crystalline Form A.

FIG. 3 is a typical DSC curve of the crystalline Form A.

FIG. 4 is a is a characteristic X-ray diffraction pattern of the crystalline Form B.

FIG. 5 is a carbon-13 cross-polarization magic-angle spinning (CPMAS) nuclear magnetic resonance (NMR) spectrum of the crystalline Form B.

FIG. 6 is a typical DSC curve of the crystalline Form B.

FIG. 7 shows scanning electron micrographs (SEM) of the crystals of Form A and Form B.

FIG. 8 shows the conversion of Form B to Form A at higher temperatures.

FIG. 9 provides detailed in situ process analytical data on the conversion of Form B to Form A at elevated temperature, namely Raman spectroscopy (uncalibrated solute concentration in green and qualitative solid phase in blue), Focused Beam Reflectance Measurement (chord counts below 10 microns).

### SUMMARY OF THE INVENTION

A crystalline form (Form A) of N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide having at least one of the following characteristics:
an X-ray powder diffraction (XRPD) spectrum having at least one peak selected from the group consisting of 8.0 (±0.2), 9.3 (±0.2) and 12.0 (±0.2) degrees 2θ;
a carbon-13 cross-polarization magic-angle spinning (CPMAS) nuclear magnetic resonance (NMR) spectrum having at least one peak selected from the group consisting of 12.41, 17.99, 20.87, 25.36, 29.24, 47.44, 57.39, 62.92, 73.13, 94.90, 96.31, 114.33, 116.23, 119.33, 120.19, 126.99, 127.85, 129.72, 133.48, 135.48, 136.67, 141.64, and 178.14 ppm; or
a differential scanning calorimetry (DSC) thermogram comprising an endothermic peak at about 181° C.

### DETAILED DESCRIPTION

N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide (MK-0674) has been found to exist in two polymeric forms, Form A and Form B.

The polymorphic Form A of MK-0674 is stable at temperatures equal and higher than 40° C. and carries lower risk of form conversion during active pharmaceutical ingredient (API) and drug product processing relative to Form B.

There are several advantages of the Form A polymorph crystals over Form B polymorph crystals.

Form A demonstrates faster crystal growth kinetics than Form B at elevated temperature which yields thicker rods of the Form A crystals. In contrast, Form B is generated at lower temperature with slower growth kinetics which produces thinner needles of Form B crystals. These thinner crystals of From B require longer filtration times which in turn results in difficulties during washing and drying of the API.

The turnover or conversion of Form A crystals to Form B crystals below 40° C. is very slow. The same is true for the conversion of Form B crystals to Form A crystals below 40° C. However, the API granulation and drying steps are performed at temperatures above 40° C. Under these conditions, Form A has no turnover risk here, whereas it has been shown that Form A phase impurities in Form B can induce turnover of Form B to Form A.

The crystalline anhydrous Forms A and B of MK-0674 were characterized by X-ray powder diffraction (XRPD), carbon-13 solid state NMR (ssNMR), and Differential Scanning calorimetry (DSC).

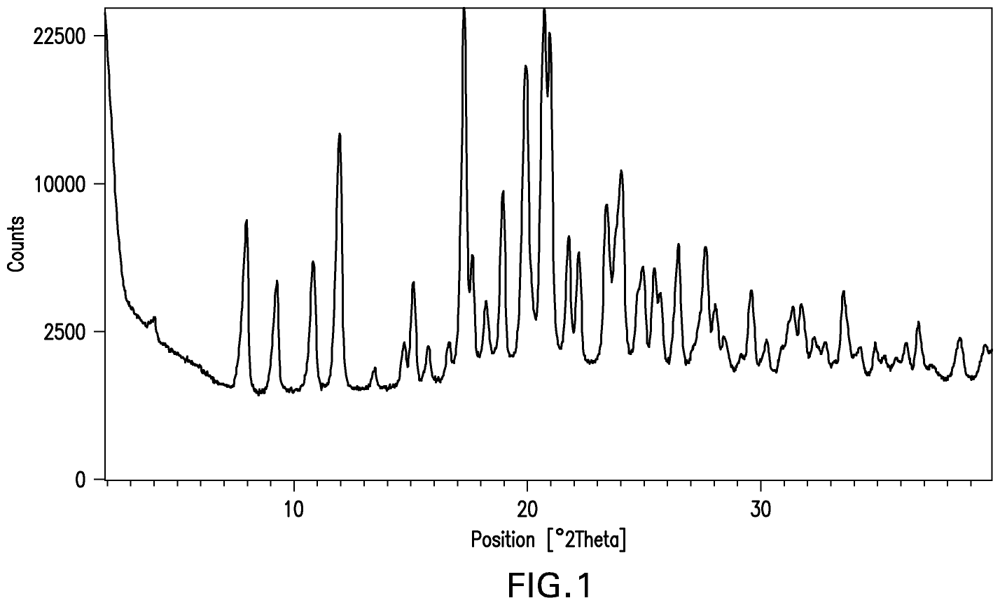

In an embodiment, the crystalline form of Form A, having an X-ray powder diffraction (XRPD) spectrum substantially as shown in FIG. 1.

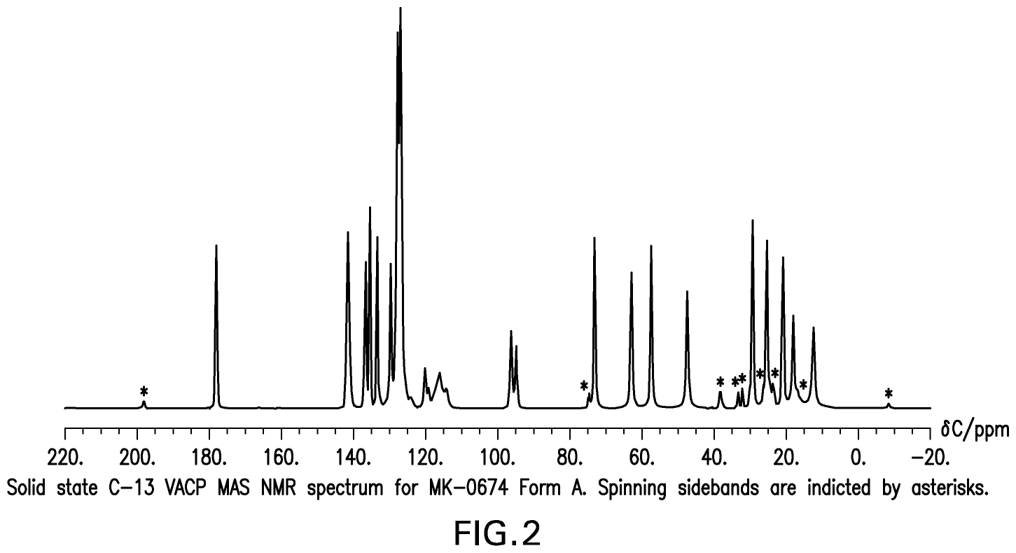

In an embodiment, the crystalline form of Form A, having carbon-13 cross-polarization magic-angle spinning (CPMAS) nuclear magnetic resonance (NMR) spectrum substantially as shown in FIG. 2.

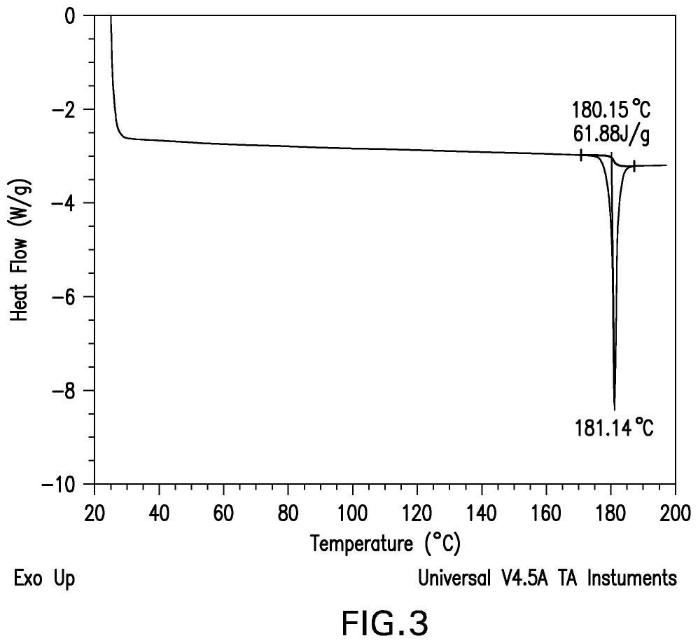

In an embodiment, the crystalline form of Form A, having a differential scanning calorimetry (DSC) thermogram substantially as shown in FIG. 3.

In an embodiment, the crystalline form of Form A of N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide having a carbon-13 cross-polarization magic-angle spinning (CPMAS) nuclear magnetic resonance (NMR) spectrum having at least one peak selected from the group consisting of Form A: 12.41, 62.92, 94.90, 133.48, 141.64, and 178.14 ppm.

In an embodiment, the crystalline form of Form A, wherein the crystalline form is thermodynamically stable at a temperature in the range of about 40° C. to about 180° C.

In an additional embodiment, a pharmaceutical composition comprising the crystalline form of Form A and a pharmaceutical excipient.

In an additional embodiment, the pharmaceutical composition of Form A, wherein the crystalline form is substantially purified.

An additional embodiment is a method of treating or preventing a cathepsin dependent disease or condition in a mammal comprising administering the composition of Form A.

In an additional embodiment, the cathepsin dependent disease or condition is osteoarthritis.

An additional embodiment is a process for preparing the crystalline form of Form A comprising precipitating the crystalline form from a solution comprising N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide and a solvent.

An additional embodiment of the process, wherein the solvent is selected from the group consisting of N.N-dimethylformamide, C₁-C₄ alkyl alcohols, water and mixtures thereof.

An additional embodiment of the process, wherein the precipitating was induced by the sequential addition of aqueous phosphoric acid and water to the solution.

An additional embodiment of the process, wherein
a) the acid is added to the solution at the temperature of above 40° C., preferably about 60° C.;
b) the water is added at the temperature of above 40° C., preferably about 50-55° C.; and
c) the resulting mixture is stirred for 2 hours at 50-55° C. before being allowed to cool to room temperature

Form B

An alternative embodiment of the invention is a crystalline form (Form B) of N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide having at least one of the following characteristics:

an X-ray powder diffraction (XRPD) spectrum having at least one peak selected from the group consisting of 9.8 (±0.2), 10.3 (±0.2) and 11.2 (±0.2) degrees 2θ;

a carbon-13 cross-polarization magic-angle spinning (CPMAS) nuclear magnetic resonance (NMR) spectrum having at least one peak selected from the group consisting of 14.34, 18.44, 20.36, 27.82, 28.77, 46.88, 57.49, 58.34, 64.09, 70.69, 72.70, 74.74, 96.06, 97.25, 121.72, 122.53, 125.48, 126.83, 127.96, 128.56, 129.29, 132.15, 132.84, 134.44, 135.26, 136.46, 137.58, 138.27, 139.01, 139.86, 140.82, 166.66, 123.48, and 176.47 ppm; or a differential scanning calorimetry (DSC) thermogram comprising an endothermic peak at about 181° C.

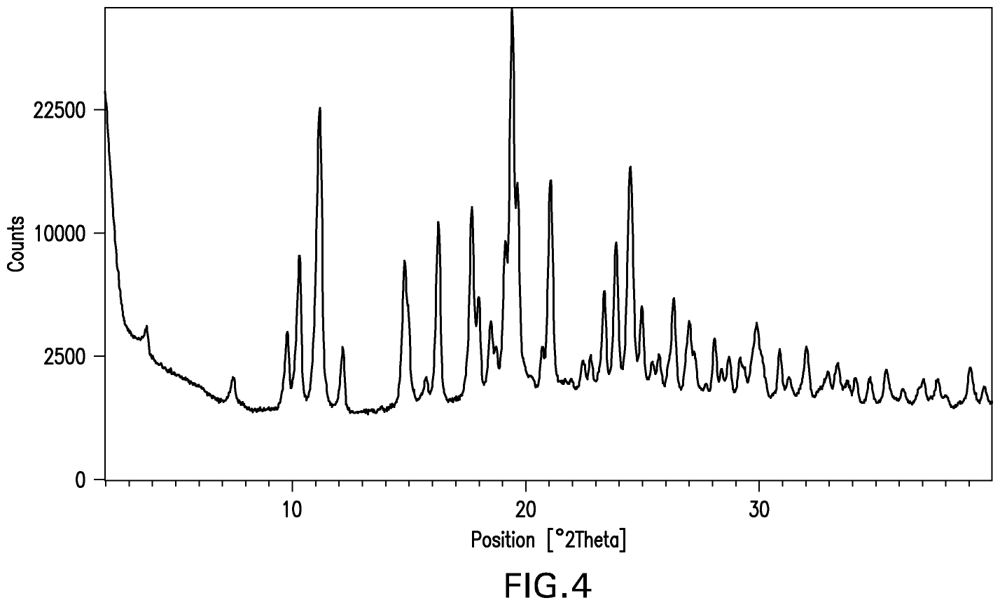

An alternative embodiment of the crystalline form of Form B, having an X-ray powder diffraction (XRPD) spectrum substantially as shown in FIG. 4.

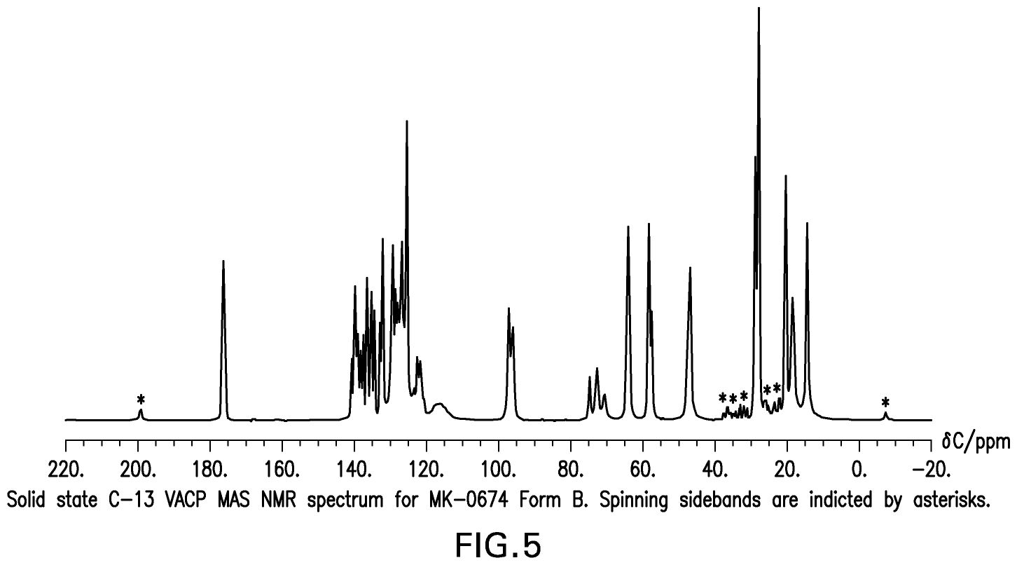

An alternative embodiment of the crystalline form of Form B, having carbon-13 cross-polarization magic-angle spinning (CPMAS) nuclear magnetic resonance (NMR) spectrum substantially as shown in FIG. 5.

An alternative embodiment of the crystalline form of Form B, having carbon-13 cross-polarization magic-angle spinning (CPMAS) nuclear magnetic resonance (NMR) spectrum having at least one peak selected from the group consisting of 14.34, 64.09, 97.25, 132.15, 139.86, and 176.47 ppm.

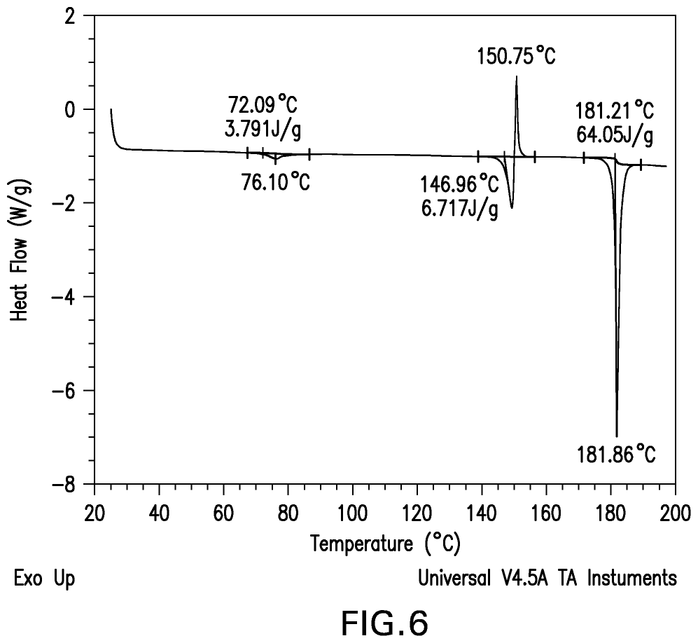

An alternative embodiment of the crystalline form of Form B having a differential scanning calorimetry (DSC) thermogram substantially as shown in FIG. 6.

An alternative embodiment is a pharmaceutical formulation comprising the crystalline form (Form A) of N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide and at least one pharmaceutically acceptable excipient.

An alternative embodiment is a pharmaceutical formulation comprising the crystalline form (Form B) of N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide and at least one pharmaceutically acceptable excipient.

### EXAMPLES

Samples of Forms A and B were prepared as follows:

Form B crystals were prepared by cooling a solution of (2S)-2-[[(1S)-1-[4-[4-[(1R)-2,2-difluoro-1-hydroxy-ethyl]phenyl]phenyl]-2,2,2-trifluoro-ethyl]amino]-4-fluoro-4-methyl-pentanoic acid (1.77 kg, 3.81 mol) in N,N-dimethylacetamide (15 L) to 0° C. Aminocyclopropanecarbonitrile hydrochloride (541 g, 4.56 mol) and 4-methylmorpholine (1.05 L, 9.54 mol) were sequentially added while keeping the temperature below 5° C. 2-(3H[1,2,3]triazolo[4,5-b]pyridin-3-yl)-1,1,3,3-tetramethylisouronium hexafluorophosphate (1.73 kg, 4.56 mol) was added under stirring to the obtained suspension and the resulting mixture was allowed to reach room temperature within 90 min to further react at this temperature for 2 hours. The reaction mixture was cooled to 0° C., diluted with isopropylacetate (28.0 L) and then aqueous 3M hydrochloric acid (8.8 L) was added. The resulting mixture was warmed to room temperature. After separation of the organic layer, the aqueous layer was extracted with isopropylacetate (12 L) and this organic phase was then washed with aqueous 3M hydrochloric acid (4.4 L). The combined organic layers were washed with aqueous 3M hydrochloric acid (6×8.8 L).

This protocol was repeated a second time and the organic phases issued from both reactions were combined to be further processed as described below.

The combined batch was concentrated to a volume of about 8 L, not exceeding an internal temperature of 35° C. The obtained concentrated solution was then diluted with methyl-tert-butylether (19.4 L) and heated to 35° C. before being cooled to a temperature of about 27° C. over 4.5 hours at which point onset of crystallization was observed. The temperature was raised to 33° C. and the thick slurry was aged for 1 hour at this temperature. While maintaining the temperature at 33° C., heptane (33 L) was added over 2.5 hours to the slurry which was aged for 1 hour. The slurry was then allowed to cool to room temperature overnight. The obtained suspension was filtered and the cake was then slurry washed with a 2:3 mixture of methyl-tert-butylether and heptane (4 L). The solid obtained was dried first by applying a nitrogen stream and then under vacuum. The obtained solid was taken up in methanol (36 L) to which Calgon ADP Carbon (2.7 kg) was added. The resulting mixture was agitated at room temperature for 3 hours and then filtered through a pad of solka floc which was rinsed with methanol (about 20 L). The filtrate was then concentrated to a volume of about 7 L while keeping the internal temperature between 21 and 23° C. Isopropylacetate (22 L) was added to the suspension which was concentrated again to a volume of about 7 L. After dilution of the suspension with methyl-tert-butylether (18 L), the temperature was raised to 35° C. The thick slurry was then cooled down to 30° C. and heptane (14 L) was added over 4 hours, while keeping the temperature between 25 and 30° C. The slurry was then allowed to reach room temperature overnight. The suspension was filtered and the cake was slurry washed with a 2:3 mixture of methyl-tert-butylether and heptane (4 L). The cake was dried first by applying a nitrogen stream and then under vacuum to afford the desired product (3.56 kg, 6.74 mol).

Form A was prepared from a crude sample of (2S)—N-(1-cyanocyclopropyl)-2-[[(1S)-1-[4-[4-[(1R)-2,2-difluoro-1-hydroxy-ethyl]phenyl]phenyl]-2,2,2-trifluoro-ethyl]amino]-4-fluoro-4-methyl-pentanamide which had been obtained by the reaction of (2S)-2-[[(1S)-1-[4-[4-[(1R)-2,2-difluoro-1-hydroxy-ethyl]phenyl]phenyl]-2,2,2-trifluoro-ethyl]amino]-4-fluoro-4-methyl-pentanoic acid (545 g, 1.18 mol) and 1-aminocyclopropanecarbonitrile hydrochloride (167 g, 1.41 mol). The temperature of the reaction mixture was increased to 60° C. over 90 min and aqueous 4% phosphoric acid (6.52 L) was added. After completion of the addition, a turbid mixture was obtained. Water (8.75 L) was added within 90 min at a temperature between 50 and 55° C. and the resulting mixture was stirred at this temperature for 2 hours. The reaction mixture was then allowed to cool to 20 to 25° C. over 18 hours. The obtained suspension was filtered, the reactor was washed with water (800 mL) which was used to rinse the cake. The cake was sequentially slurry washed with a 1 to 3 mixture of N,N-dimethylformamide and water (1.5 L) and then with water (3×3 L) before being dried by applying a nitrogen flow to afford the desired product as white solid (610 g, 1.16 mol).

Each of these samples of Forms A and B were characterized as described below:

The X-Ray Powder Diffraction (XRPD)

X-ray powder diffraction studies are widely used to characterize molecular structures, crystallinity, and polymorphism. The X-ray powder diffraction patterns of Form A and Form B were generated on Bruker AXS D8 Advance with a LYNXEYE XE-T detector in reflection mode.

Solid State NMR

In addition to the X-ray powder diffraction patterns described above, Form A and Form B samples were further characterized based on their carbon-13 solid-state nuclear magnetic resonance (NMR) spectrum. The carbon-13 spectrum was recorded on a Bruker AVANCE III NMR spectrometer operating at 500.13 MHz, using a Bruker 4 mm H/X/Y triple resonance CPMAS probe. The spectrum was collected utilizing proton/carbon-13 variable-amplitude cross-polarization (VACP) at 83.3 kHz, with a contact time of 3 ms. Other experimental parameters used for data acquisition were a proton 90-degree pulse of 100 kHz, high-power proton TPPM decoupling at 100 kHz, a pulse delay of 1.6 s, a dwell time of 5.0 μs, an acquisition time of 20.48 ms, and signal averaging for 17000 scans. A magic-angle spinning (MAS) rate of 13 kHz was used for data collection. A Lorentzian line broadening of 30 Hz and zero filling to 32768 points were applied to the spectrum before Fourier Transformation. Chemical shifts are reported on the TMS scale using the carbonyl carbon of glycine (176.70 ppm) as a secondary reference.

Differential Scanning calorimetry (DSC)

DSC data were acquired using TA Instruments DSC Q2000 or equivalent instrumentation. A sample with a weight between 1 and 6 mg was weighed into an open pan. This pan was placed in the sample position in the calorimeter cell. An empty pan was placed in the reference position. The calorimeter cell was closed and a flow of nitrogen passed through the cell. The heating program was set to heat the sample at a heating rate of 10° C./min to a temperature of approximately 200° C. When the run was completed, the data were analyzed using the DSC analysis program in the system software. The observed endo- and exotherms were integrated between baseline temperature points that are above and below the temperature range over which the endotherm is observed. The data reported are the onset temperature, peak temperature and enthalpy.

Physical Characterization of MK-0674 Crystalline Form A

FIG. 1 shows the X-ray powder diffraction pattern of MK-0674 Form A. Form A exhibited characteristic diffraction peaks corresponding to d-spacings of 11.1, 9.5, and 7.4 angstroms. Form A was further characterized by the d-spacings of 8.2, 5.1, and 4.4 angstroms. Form A was even further characterized by the d-spacings of 4.1, 4.0, and 3.2 angstroms.

TABLE 1
Characteristic Peak Position and
Corresponding d-Spacing for Form A

| Peak | d- |
|---|---|
| Position | Spacing |
| [°2θ] | [Å] |
| 4.0 | 22.1 |
| 8.0 | 11.1 |
| 9.3 | 9.5 |
| 10.9 | 8.2 |
| 12.0 | 7.4 |
| 13.5 | 6.6 |
| 14.7 | 6.0 |
| 15.1 | 5.9 |
| 15.7 | 5.6 |
| 16.6 | 5.3 |
| 17.3 | 5.1 |
| 17.6 | 5.0 |
| 18.2 | 4.9 |
| 18.9 | 4.7 |
| 20.0 | 4.4 |
| 20.7 | 4.3 |
| 21.0 | 4.2 |
| 21.8 | 4.1 |
| 22.2 | 4.0 |
| 23.3 | 3.8 |
| 24.0 | 3.7 |
| 24.7 | 3.6 |
| 24.9 | 3.6 |
| 25.4 | 3.5 |
| 25.7 | 3.5 |
| 26.5 | 3.4 |
| 27.6 | 3.2 |
| 28.0 | 3.2 |
| 28.4 | 3.1 |
| 29.1 | 3.1 |
| 29.6 | 3.0 |
| 30.3 | 3.0 |
| 30.8 | 2.9 |
| 31.4 | 2.9 |
| 31.7 | 2.8 |
| 32.2 | 2.8 |
| 32.8 | 2.7 |
| 33.5 | 2.7 |
| 34.3 | 2.6 |
| 34.9 | 2.6 |
| 35.3 | 2.5 |
| 35.8 | 2.5 |
| 36.2 | 2.5 |
| 36.7 | 2.4 |
| 37.3 | 2.4 |
| 38.5 | 2.3 |
| 39.5 | 2.3 |

FIG. 2 shows the carbon-13 cross-polarization magic-angle spinning (CPMAS) nuclear magnetic resonance (NMR) spectrum of Form A. Characteristic peaks for Form A are observed at 12.41, 17.99, 20.87, 25.36, 29.24, 47.44, 57.39, 62.92, 73.13, 94.90, 96.31, 114.33, 116.23, 119.33, 120.19, 126.99, 127.85, 129.72, 133.48, 135.48, 136.67, 141.64, and 178.14 ppm.

FIG. 3 is a typical DSC curve of the crystalline Form A ((NB-xjin2-0385446-0022). The DSC curve is characterized by a melting endotherm with an extrapolated onset temperature of 180.2° C., a peak temperature of 181.1° C. and enthalpy of 61.9 J/g.

Physical Characterization of MK-0674 Crystalline Form B

FIG. 4 shows the X-ray powder diffraction pattern of MK-0674 Form B. Form B exhibited characteristic diffraction peaks corresponding to d-spacings of 9.0, 8.6, and 7.9 angstroms. Form B was further characterized by the d-spacings of 5.5, 4.6, and 3.6 angstroms.

TABLE 2
Characteristic Peak Position and
Corresponding d-Spacing for Form B

| Peak | d- |
|---|---|
| Position | Spacing |
| [°2θ] | [Å] |
| 3.8 | 23.5 |
| 7.5 | 11.8 |
| 9.8 | 9.0 |
| 10.3 | 8.6 |
| 11.2 | 7.9 |
| 12.2 | 7.3 |
| 14.8 | 6.0 |
| 15.0 | 5.9 |
| 15.7 | 5.6 |
| 16.2 | 5.5 |
| 17.7 | 5.0 |
| 18.0 | 4.9 |
| 18.5 | 4.8 |
| 18.7 | 4.7 |
| 19.1 | 4.6 |
| 19.4 | 4.6 |
| 19.6 | 4.5 |
| 20.7 | 4.3 |
| 21.0 | 4.2 |
| 21.9 | 4.0 |
| 22.4 | 4.0 |
| 22.8 | 3.9 |
| 23.4 | 3.8 |
| 23.9 | 3.7 |
| 24.5 | 3.6 |
| 25.0 | 3.6 |
| 25.4 | 3.5 |
| 25.7 | 3.5 |
| 26.3 | 3.4 |
| 27.0 | 3.3 |
| 27.2 | 3.3 |
| 27.7 | 3.2 |
| 28.1 | 3.2 |
| 28.4 | 3.1 |
| 28.7 | 3.1 |
| 29.2 | 3.1 |
| 29.9 | 3.0 |
| 30.9 | 2.9 |
| 31.2 | 2.9 |
| 32.0 | 2.8 |
| 32.9 | 2.7 |
| 33.4 | 2.7 |
| 33.8 | 2.7 |
| 34.1 | 2.6 |
| 34.7 | 2.6 |
| 35.4 | 2.5 |
| 36.2 | 2.5 |
| 37.1 | 2.4 |
| 37.6 | 2.4 |
| 38.0 | 2.4 |
| 39.0 | 2.3 |
| 39.6 | 2.3 |

FIG. 5 shows the carbon-13 cross-polarization magic-angle spinning (CPMAS) nuclear magnetic resonance (NMR) spectrum of Form B. Characteristic peaks for Form B are observed at 14.34, 18.44, 20.36, 27.82, 28.77, 46.88, 57.49, 58.34, 64.09, 70.69, 72.70, 74.74, 96.06, 97.25, 121.72, 122.53, 125.48, 126.83, 127.96, 128.56, 129.29, 132.15, 132.84, 134.44, 135.26, 136.46, 137.58, 138.27, 139.01, 139.86, 140.82, 166.66 (very broad), 123.48, and 176.47 ppm.

FIG. 6 is a typical DSC curve of the crystalline Form B ((NB-xjin2-0385446-0022). The DSC curve is characterized by three endotherms and one exotherm. The first endotherm with an extrapolated onset temperature of 72.1° C., a peak temperature of 76.1° C. and enthalpy of 3.8 J/g is due to polymorphic transition to Form C. The endotherm with an extrapolated onset temperature of 147.0° C. is due to melting of Form C. The exotherm with a peak temperature of 150.8° C. is due to crystallization of Form A from the melt. The endotherm with an extrapolated onset temperature of 181.2° C., a peak temperature of 181.9° C. and enthalpy of 64.1 J/g is due to melting of Form A.

Relative Thermodynamic Stability of Form A and Form B

Form A and Form B are enantiotropically related. Competitive slurry experiments of Forms A and B in ethanol/water at 25° C., 30° C., 35° C. and 40° C. were used to establish the transition temperature of the enantiotropic forms. Form A is the more stable form at temperatures equal or higher than 40° C., while and Form B is more stable at temperatures equal or lower than 30° C.

Stability of Form A and Form B During Processing

Form A does not convert to Form B during timeframes which are typical for API process and DP processing in the temperature range where Form B is stable due to slow crystal growth kinetics of Form B and limited driving force, i.e., the solubility difference between the two forms. Contrarily, Form B converts to Form A in the process solvent above 50° C. in few hours in the absence of Form A seeds. Thus, there is a potential risk of Form B conversion to Form A during a typical wet granulation process, when seeds of Form A are present. Based on the kinetics of form conversion studies it was concluded that the crystallization process designed to deliver Form A, as well as wet granulation using Form A carries lower risk of form conversion compared to those processes where Form B is used.

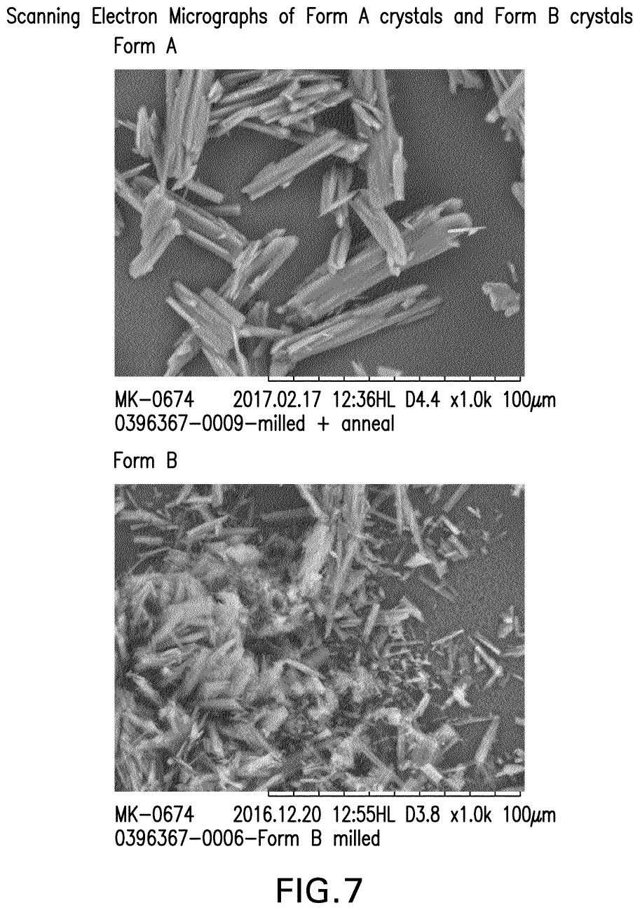

FIG. 7 shows scanning electron micrographs (SEM) of the crystals of Form A and Form B. These micrographs were taken after milling the crystals as a suspension in the isolation solvents using a rotor-stator mill. The lower aspect ratio and larger size of the Form A crystals facilitates solid-liquid separation and results in superior flow properties compared to the smaller needle like crystals of Form B.

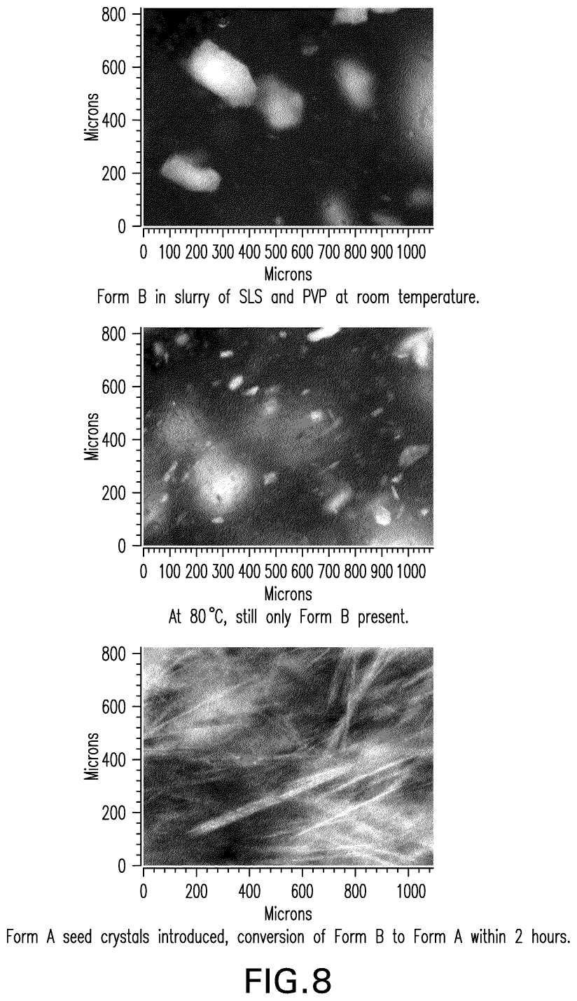

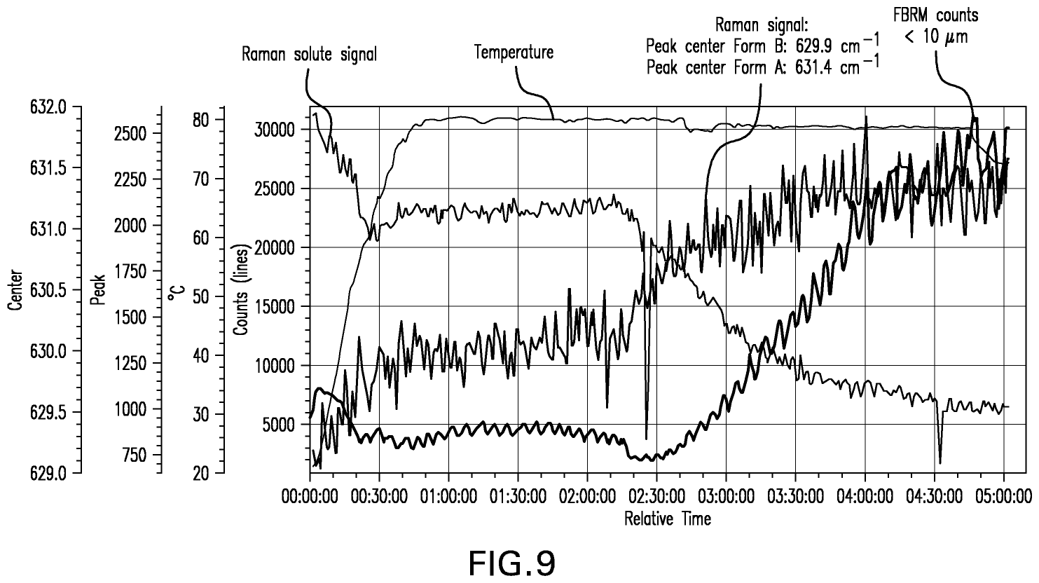

FIG. 8 shows the results of an experiment to test the conversion of Form B to Form A at 80° C. in a slurry of aqueous sodium lauryl sulfate (SLS) and polyvinyl pyrrolidone (PVP). The Form B crystals were added to the SLS and PVP to form a slurry. The temperature of the slurry was raised to 80° C. with no Form A detected. Once Form A crystal seeds were introduced, most of the Form B crystals were converted to Form A crystals within 2 hours. FIG. 9 depicts complimentary in situ process analytical data from Raman spectroscopy (on solute concentration and solid phase composition) and Focused Beam Reflectance Measurement (FBRM characterizing number and dimension of the dispersed particles). It can be readily observed that during the initial heat-up phase and the subsequent isothermal phase until about 2h 15 min (marked with a small red triangle), all trends from Raman spectroscopy and FBRM are roughly constant. This indicates that the dispersed Form B particles do not change in this period. However, upon addition of Form A seeds at around 2h 15 min (marked with the small triangle) the solute signal drops over time whereas FBRM counts as well as the trend characterizing the suspended solid form increase. Thus, all trends indicate a form conversion from Form B to Form A: The Raman solute signal drops in this phase due to the lower solubility of Form A, the Raman signal characterizing the solid form undergoes a peak shift from 629.9 cm⁻¹ to 631.4 cm⁻¹ and the FBRM counts increase due to the nucleation and growth of Form A crystals as shown in FIG. 8. Thus, it can be concluded that traces of Form A are sufficient to induce form conversion of Form B to Form A at elevated temperatures.

1. A crystalline form of N′-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide having at least one of the following characteristics:
an X-ray powder diffraction (XRPD) spectrum having at least one peak selected from the group consisting of 8.0 (±0.2), 9.3 (±0.2) and 12.0 (±0.2) degrees 2Θ;
a carbon-13 cross-polarization magic-angle spinning (CPMAS) nuclear magnetic resonance (NMR) spectrum having at least one peak selected from the group consisting of 12.41, 17.99, 20.87, 25.36, 29.24, 47.44, 57.39, 62.92, 73.13, 94.90, 96.31, 114.33, 116.23, 119.33, 120.19, 126.99, 127.85, 129.72, 133.48, 135.48, 136.67, 141.64, and 178.14 ppm; or
a differential scanning calorimetry (DSC) thermogram comprising an endothermic peak at about 181° C.

2. The crystalline form of claim 1, having an X-ray powder diffraction (XRPD) spectrum substantially as shown in FIG. 1.

3. The crystalline form of claim 1, having carbon-13 cross-polarization magic-angle spinning (CPMAS) nuclear magnetic resonance (NMR) spectrum substantially as shown in FIG. 2.

4. The crystalline form of claim 1, having a differential scanning calorimetry (DSC) thermogram substantially as shown in FIG. 3.

5. A crystalline form of N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide having a carbon-13 cross-polarization magic-angle spinning (CPMAS) nuclear magnetic resonance (NMR) spectrum having at least one peak selected from the group consisting of Form A: 12.41, 62.92, 94.90, 133.48, 141.64, and 178.14 ppm.

6. The crystalline form of claim 1, wherein the crystalline form is thermodynamically stable at a temperature in the range of about 40° C. to about 180° C.

7. A pharmaceutical composition comprising the crystalline form of claim 1 and a pharmaceutical excipient.

8. The pharmaceutical composition of claim 7, wherein the crystalline form is substantially purified.

9. A method of treating or preventing a cathepsin dependent disease or condition in a mammal comprising administering the composition of claim 7.

10. The method of claim 9, wherein the cathepsin dependent disease or condition is osteoarthritis.

11. A process for preparing the crystalline form of claim 1 comprising precipitating the crystalline form from a solution comprising N¹-(1-cyanocyclopropyl)-N²-((1S)-1-{4′-[(1R-2,2-difluoro-1-hydroxyethyl]biphenyl-4-yl}-2,2,2-trifluoroethyl)-4-fluoro-L-leucinamide and a solvent.

12. The process of claim 11, wherein the solvent is selected from the group consisting of N,N-dimethylformamide, C₁-C₄ alkyl alcohols, water and mixtures thereof.

13. The process of claim 11, wherein the precipitation was induced by the sequential addition of aqueous phosphoric acid and water to the solution.

14. The process of claim 13, wherein
a) the acid is added to the solution at the temperature of above 40° C., preferably about 60° C.;
b) the water is added at the temperature of above 40° C., preferably about 50-55° C.; and
c) the resulting mixture is stirred for 2 hours at 50-55° C. before being allowed to cool to room temperature.

15. A pharmaceutical composition comprising the crystalline form of claim 2 and a pharmaceutical excipient.

16. A pharmaceutical composition comprising the crystalline form of claim 3 and a pharmaceutical excipient.

17. A pharmaceutical composition comprising the crystalline form of claim 4 and a pharmaceutical excipient.

18. A pharmaceutical composition comprising the crystalline form of claim 5 and a pharmaceutical excipient.

19. A method of treating or preventing a cathepsin dependent disease or condition in a mammal comprising administering the composition of claim 15.

20. A method of treating or preventing a cathepsin dependent disease or condition in a mammal comprising administering the composition of claim 16.
